(19) United States
(12) Patent Application Publication
(10) Pub. No.: US 2016/0333390 A1
(43) Pub. Date: Nov. 17, 2016

(54) LOW COST GLUCOSE MONITORING SYSTEM AND METHOD OF USE

(71) Applicant: Clemson University, Clemson, SC (US)

(72) Inventors: Kayla Elizabeth Gainey, Seneca, SC (US); Patrick Tyler Ovington, Greenville, SC (US); Delphine Marguerite Dean, Central, SC (US); Laura Kaitlin Wiles, Philadelphia, PA (US); Reba Maglin Halsey, Greenville, SC (US); Suzanna Langworthy, Flower Mound, TX (US)

(21) Appl. No.: 14/709,700

(22) Filed: May 12, 2015

(51) Int. Cl. C12Q 1/54; G01N 21/78

(52) U.S. Cl. CPC C12Q 1/54; G01N 21/78; G01N 2201/062; G01N 2021/7753

(57) ABSTRACT

An improved system for monitoring blood glucose, especially in remote regions, is provided. The system includes a substrate comprising a sample umbra and a control umbra and a printer capable of printing coatings on the substrate thereby forming a printed substrate. A first coating comprises glucose oxidase and the first coating is in the sample umbra but not in the control umbra. A second coating comprises peroxidase and the second coating is in the sample umbra and the control umbra. A third coating comprises and indicator and the third coating is in the sample umbra and the control umbra. A barrier encases at least a portion of the coated substrate except for a sample window. When blood is applied to the sample window plasma of the blood wicks into the substrate, exclusive of red blood cells, such that the plasma enters the sample umbra and the control umbra. A detector is provided which is capable of measuring a color change of the indicator in the sample umbra and the control umbra.

### BACKGROUND

The present invention is related to a low cost system for monitoring glucose and a method of using the system. More specifically, the present invention is related to an improved system, and method for using the system, wherein the system is suitable for use in third world countries lacking adequate storage and medical infrastructure and in areas where access to health care is limited.

Diabetes is a significant health concern across the world. Though a cure for diabetes is widely sought, the current medical approach requires monitoring and controlling the glucose levels through diet and/or insulin administration. Test procedures have developed to the point where it is no longer necessary for diabetes to be a debilitating disease and, in fact, most people in developed nations can live quite a normal life with diabetes. Test kits and insulin are widely available for most in the developed nations and the infrastructure is sufficient to support most diabetics.

In the underdeveloped nations quite a different situation exist. In many parts of the world test supplies are virtually unavailable and even those with the greatest of intentions are thwarted by the long transit time required to get test supplies to those that need it the most. It is not uncommon for test supplies to expire long before they can reach the intended patient. Furthermore, the cost associated with the manufacture and shipping of conventional test supplies insures that only those with benevolent intent even attempt to supply the need rendering the available supply neither sufficient nor sustainable.

There is a significant need for test supplies for blood plasma based measurements, and particularly for measuring glucose or blood sugar, which has extremely long shelf lives and which can be delivered to a remote village clinic, or remote diabetic, at a minimal cost. This need has currently not been met. The present invention satisfies this need.

### SUMMARY OF THE INVENTION

It is an object of the invention to provide a system, and method, for testing glucose wherein components of the system can be prepared on site thereby minimizing degradation over time and the system can be implemented using inexpensive, readily available, technologies.

It is another object of the invention to provide a system, and method, for testing glucose which is auto-calibrating and which does not require preservatives.

These and other advantages, as will be realized, are provided in a system for measuring glucose in blood. The system includes a substrate comprising a sample umbra and a control umbra and a printer capable of printing coatings on the substrate thereby forming a printed substrate. A first coating comprises glucose oxidase and the first coating is in the sample umbra but not in the control umbra. A second coating comprises peroxidase, preferably horseradish peroxidase, and the second coating is in the sample umbra and the control umbra. A third coating comprises and indicator and the third coating is in the sample umbra and the control umbra. A barrier encases at least a portion of the coated substrate except for a sample window. When blood is applied to the sample window plasma of the blood wicks into the substrate, exclusive of red blood cells, such that the plasma enters the sample umbra and the control umbra. A detector is provided which is capable of measuring a color change of the indicator in the sample umbra and the control umbra.

Yet another embodiment of the invention is provided in A method for testing glucose in blood comprising:
providing a substrate comprising a sample umbra and a control umbra;
providing a printer capable of applying coatings on the substrate wherein the printer comprises cartridges wherein:
a first cartridge of the cartridges comprises a first liquid wherein the first liquid comprises glucose oxidase;
a second cartridge of the cartridges comprises a second liquid wherein the second liquid comprises peroxidase, preferably horseradish peroxidase; and
a third cartridge of the cartridges comprises a third liquid wherein the third liquid comprises indicator, preferably a colorimetric indicator;
forming a printed substrate by:
applying the first liquid to the substrate thereby forming a first coating wherein the first coating is in the sample umbra but not in the control umbra;
applying the second liquid to the substrate thereby forming a second coating wherein the second coating is in the sample umbra and in the control umbra;
applying the third liquid to the substrate thereby forming a third coating wherein the third coating is in the sample umbra and in the control umbra;
encasing the printed substrate with a barrier except for a sample window wherein the barrier covers the sample umbra and in the control umbra thereby forming an encased substrate;
applying blood to the encased substrate at the sample window;
measuring a sample color change in the sample umbra and in a control color change in the control umbra; and
subtracting the control color change from the sample color change to provide a resulting color change wherein the resulting color change is proportional to the glucose in the blood.

### FIGURES

FIG. 1 is a cross-sectional schematic view of an embodiment of the invention.

FIG. 2 is a cross-sectional schematic view of an embodiment of the invention.

FIG. 3 is a cross-sectional schematic view of an embodiment of the invention.

FIG. 4 is a flow chart representation of an embodiment of the invention.

FIG. 5 is a top perspective schematic view of an embodiment of the invention.

FIG. 6 is a schematic representation of an embodiment of the invention.

### DESCRIPTION

The instant invention is specific to a system for monitoring blood plasma components, particularly glucose, and a method of use. The inventive system is stable for long periods of time, is economical and can be used by an individual or clinician with minimal training. More specifically, the present invention is specific to a glucose monitoring system which can be prepared just prior to use using conventional equipment such as ink jet printers and commercially available substrates at the time of need or just before use. More specifically, the system can be transported to remote areas and then assembled by a clinician for distribution to users or by the user themselves. A particular feature is the lack of dependence on preservatives.

The invention will be described with reference to the various figures which form an integral non-limiting component of the disclosure. Throughout the disclosure similar elements will be numbered accordingly.

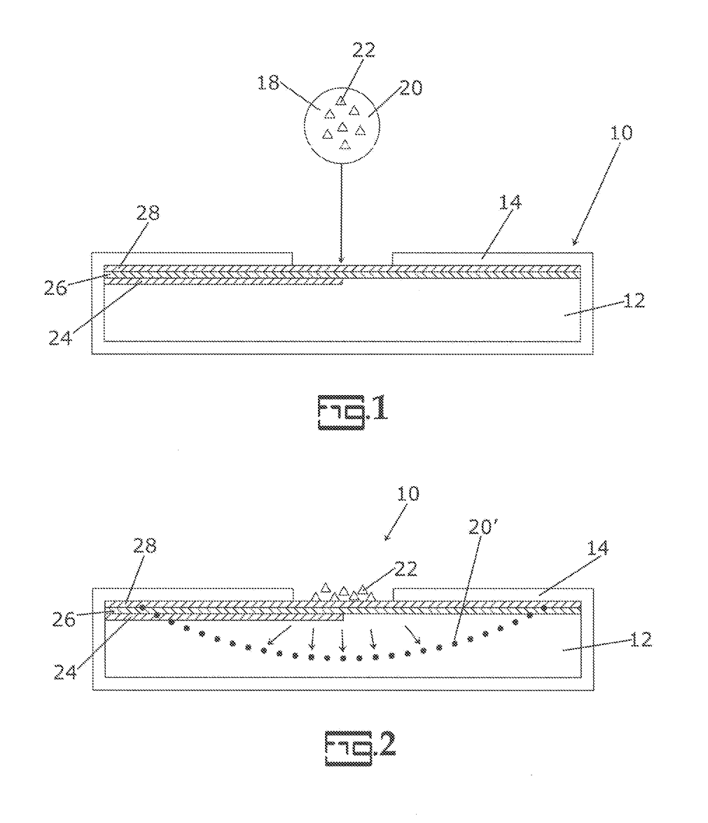

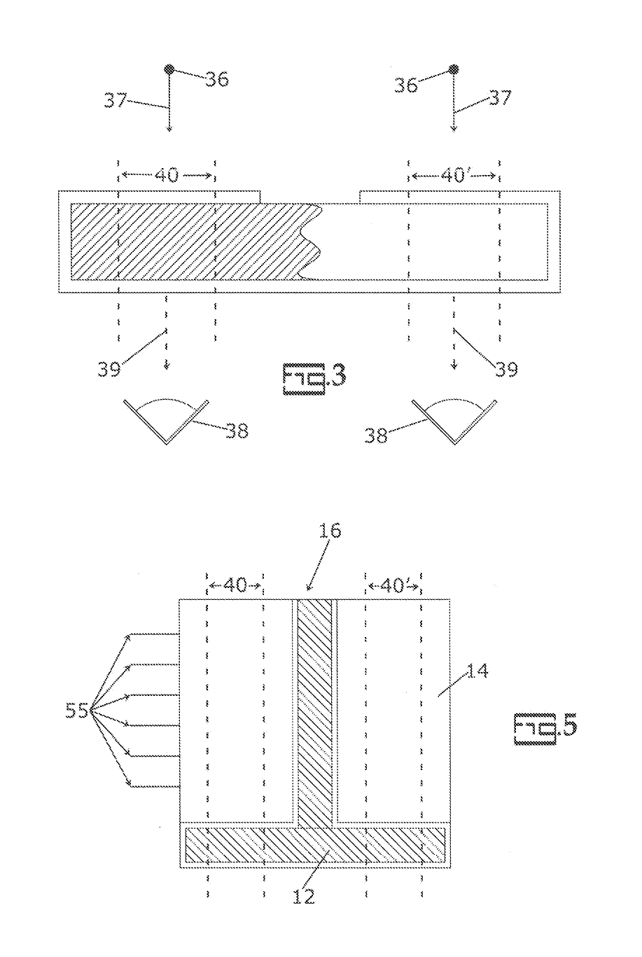

An embodiment of the invention is illustrated in FIG. 1. In FIG. 1, a test substrate is illustrated in schematic cross-sectional view as 10. The test substrate comprises a substrate, 12, with a series of coatings thereon as will be described further herein. A barrier, 14, partially encases the substrate preferably with the entire substrate being encased except for a sample window, 16, wherein sample, 18, can be inserted. The sample, 18, comprises red blood cells, 22, in plasma, 20. When the sample is applied onto the sample window, 16, of the substrate, as illustrated in schematic cross-sectional view in FIG. 2, the red blood cells are separated from the plasma and the plasma wicks into the substrate, as represented by a migration front, 20′, in the direction indicated by the arrows, and any glucose in the plasma reacts with materials coated thereon as will be more fully described.

With further reference to FIGS. 1 and 2, the substrate, 12, comprises a series of coatings thereon. While illustrated as discreet layers in a particular order it is hypothesized that the coatings permeate into the substrate and intermingle such as to be indistinguishable, at least at the interface if an interface exist. The coatings are defined as first coating, second coating and third coating for convenience without limit to the order of application even though the nomenclature does indicate the preferred order of application and the figures are illustrated schematically as such for convenience without limit thereto. The first coating, 24, is a partial coating of glucose oxidase. Glucose oxidase is known for the determination of free glucose in body fluids. Glucose and glucose oxidase react to liberate hydrogen peroxide and D-gluconic acid stoichiometrically. Those portions of the substrate which are coated with glucose oxidase are referred to herein as the sample region and those portions of the substrate which are not coated with glucose oxidase are referred to herein as a control region as will be more fully described herein.

A second coating, 26, is preferably a full coating in that the coating covers at least the sample region and the control region of the substrate. The second coating is a peroxidase layer comprising a peroxidase which reacts stoichiometrically with the hydrogen peroxide liberated from the reaction of the glucose and glucose oxidase. A particularly preferred peroxidase is horseradish peroxidase. More preferably the peroxidase is selected from the group consisting of horseradish peroxidase Type I, horseradish peroxidase Type II and horseradish peroxidase Type VI. Most preferably the peroxidase is horseradish peroxidase Type VI-A

A third coating, 28, is preferably also a full coating in that the coating covers at least the sample region and the control region of the substrate. The third coating is an indicator layer comprising an indicator capable of reacting with the hydrogen peroxide in the presence of the peroxidase to provide a color change, having an intensity which is proportional to the hydrogen peroxide, and therefore the color change is also proportional to the glucose concentration in the original sample. A particularly preferred indicator is selected from the group consisting of O-dianisidine; 3,3′,5,5′-tetramethylbenzidine; o-phenylene-diamine; 5-aminosalicylic acid and 2,2′-azino-bis(3-ethylbenzothizoline-6-sulphonic acid). A particularly preferred indicator is 2,2′-azino-bis(3-ethylbenzothizoline-6-sulphonic acid) which is referred to as ABTS.

An embodiment of the invention will be described with reference to FIG. 3 wherein a treated test substrate, 30, is illustrated in cross-sectional schematic view after application of the blood sample. A sample region, 32, is illustrated as that portion of the substrate wherein the first coating was applied and therefore the glucose of the blood plasma would react, if present, with glucose oxidase to form a colorimetric change in the indicator due to the reaction of the liberated hydrogen peroxide with the peroxidase. A control region, 34, is that portion of the substrate not coated with the first coating and therefore the components of the sample region are present except for the glucose oxidase including: the blood plasma, second coating and third coating. Any oxidation caused by environmental factors, defined as oxidation not the result of the reaction of glucose with glucose oxidase, would be equally present in both the sample region and the control region. The colorimetric change in the sample region would therefore have two components with one component being the result of glucose and the other being environmental factors. By subtraction the colorimetric change of the control section can be removed from the colorimetric change in the sample section thereby determining the colorimetric changes in the sample section resulting solely from the glucose reaction.

With further reference to FIG. 3, optical emitters, 36, pass light, 37, through both the sample region and the control region wherein the light is attenuated proportional to the colorimetric change. The attenuated light, 39, is then detected by sensors, 38, capable of quantifying the light and reporting either the light transmitted or, by difference, the light absorbance. The sample region and control region are larger than the sample umbra, 40, and control umbra, 40′, which are preferably the same size. The umbra is that portion of the substrate through which the light that is measured passes through. As would be realized the optical emitters and sensors must be matched, or calibrated, or a common optical emitter and/or sensor can be used with optical systems incorporated to pass the light through the appropriate umbra. It is preferable to use separate optical emitters, with one for the sample region and one for the control region, and separate sensors. It is preferable to provide a calibration strip or an electronic calibration system for insuring the accuracy of the detector. With two lights and detectors it is preferable to have an electronic calibration wherein the lights and detectors are balanced electronically. It is most preferred to measure transmittance for simplicity in operation.

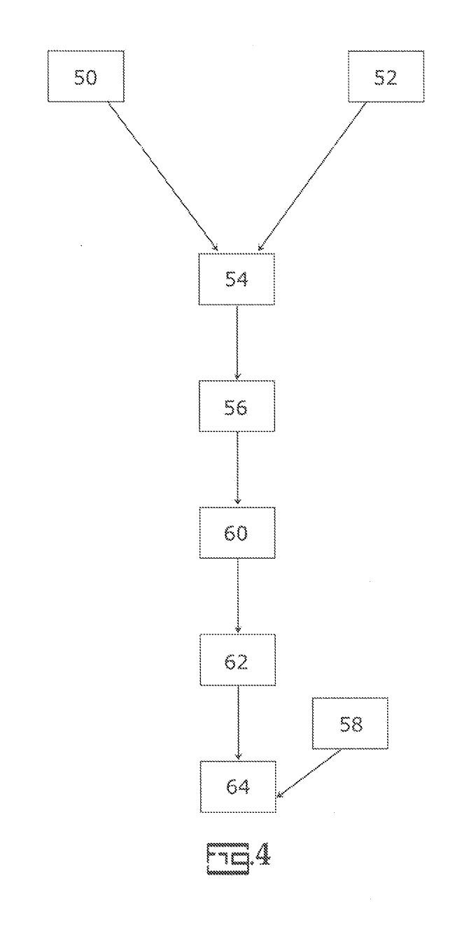

An embodiment of the invention is illustrated in flow chart form in FIG. 4 and in the form of a system in FIG. 6. A substrate, 60, is provided at 50. The substrate is preferably a sheet from which multiple test substrates will be prepared. For reasons which will be more apparent from further discussions, the size of the substrate is preferably consistent with commercially available paper sizes since this allows the use of conventional printers without modification of the feed mechanism.

A printer, 62, is prepared at 52. The printer is preferably an ink jet style printer with fillable, preferably removable, cartridges, 63-65, wherein each cartridge is designed to print independent of the others as typically employed with a color ink jet printer. For the purposes of the instant invention the cartridges are either purchased empty or emptied of the ink. Each cartridge is filled with a coating solution wherein one is a glucose oxidase solution, one is a peroxidase solution and one is an indicator solution. If the printer is equipped with four cartridges one may contain conventional ink for printing text or images on the substrate. It is particularly preferable to print instructions of use on the substrate. Many commercial printers have four colors selectable between, black, cyan, magenta and yellow. The cartridges can be provided with the coating solution contained therein, however, this leads to a limited shelf life and is not desirable. It is preferable that each cartridge be supplied in concert with a powder tube, 66, containing the active component as a lyophilized powder or, more preferably, with the cartridge pre-charged and provided with the lyophilized powder therein. Glucose oxidase, peroxidases and indicators are typically stable for a very long time as a powder thereby eliminating the need for preservatives and the like. Each cartridge is preferably supplied in concert with a water tube, 68, comprising the amount of water necessary to fill the cartridge and dissolve the powder. A syringe can be supplied for introduction of the water to the cartridge or the water may be supplied in a container which allows the water to be directly introduced into the cartridge from the container. If the powder is supplied separately from the cartridge the water and powder can be mixed prior to adding to the cartridge. The water may be provided in single use containers with each container providing only a sufficient amount of water to fill one cartridge, which is preferred, or the water may be provided in a container comprising a sufficient amount of water for multiple applications. If surfactants or other adjuvants are desired a separate water container for each cartridge is preferred. While not limited thereto, it is preferable to maintain consistency and therefore for the purposes of discussion, and as a preferred practice, the magenta cartridge is used for glucose oxidase, the cyan cartridge for peroxidase and the yellow cartridge for the indicator. This allows the color schemes, and internal controls of the printer, to be used to insure proper cartridge placement and for monitoring fill level without modification of the printer. This also allows a simple color system to be employed for supply of materials with all materials related to the glucose oxidase being labeled with magenta, for example.

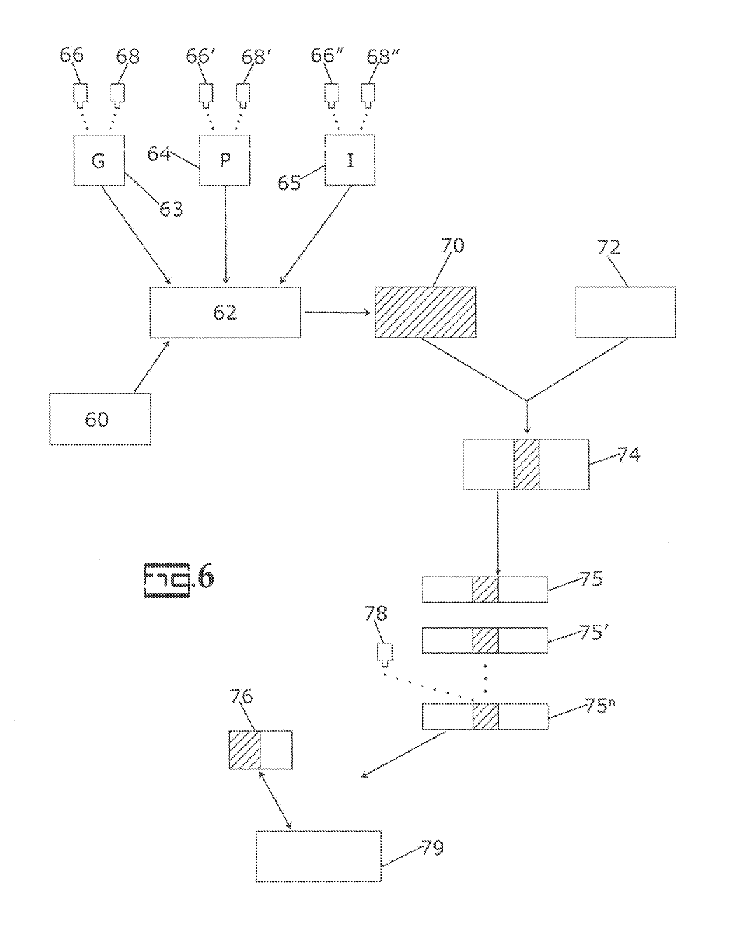

The solutions are printed on the substrate at 54 thereby forming a printed substrate, 70, using a standard word processor with the individual solutions selected using the RGB color selectors from the word processor. As would be realized the glucose oxidase solution is printed on less than the entire sheet. The control umbra, at least, must not be coated with glucose oxidase as set forth above. With reference to FIG. 5, at least the sample umbra, 40, is coated with glucose oxidase solution but not the control umbra, 40′. For simplicity, half of the substrate, can be coated with glucose oxidase. The peroxidase and indicator must cover at least the sample umbra and control umbra and may cover the entire sheet.

After printing the substrate is covered, except for the sample window, 16, with the barrier, 72, at 56 thereby forming a master test substrate, 74. The barrier is preferably a contact paper and is preferably applied to the back side of the master test substrate and folded over to the front side with the sample window thereon. It is preferable to use a straight edged tool, or similar device, to press the barrier securely to the filter paper followed by trimming any barrier from the edges. The substrate may then be cut into strips such as along cut lines, 55, thereby providing a multiplicity of test substrates, 75.

If necessary, a control strip, 76, is measured at 58 to either confirm that the light emitters and detectors are balanced or to make corrections if not. As appropriate the control strip may be measured repeatedly until balance is confirmed. After calibration the test substrate is introduced into the reader wherein light is passed through the sample umbra and control umbra as indicated above and the glucose concentration is reported in units of convenience.

A blood sample, 78, is taken from the patient at 60 using standard techniques which are well known to those of skill in the art and not amended herein.

Blood is applied to the substrate at the sample window at 62 with a sufficient volume for the plasma to wick into the substrate to the degree necessary for the sample umbra and control umbra to be equally filled. It is preferable that the plasma wick well beyond the umbra to insure adequate concentration in the umbra. As set forth above the red blood cells are excluded from the sample umbra and control umbra.

The substrate is selected from a material which will allow blood plasma to wick therein while excluding red blood cells, preferably by size exclusion. Paper is a preferred substrate due to the ready availability and low cost. A particularly preferred paper has an average pore size sufficient to exclude red blood cells from the blood plasma. A pore size of no more than 10 μm is sufficient to demonstrate the invention. Above a pore size of about 10 μm the size exclusion may be insufficient. It is more preferable to have a paper with a pore size of no more than about 7.5 μm and even more preferably no more than about 5 μm. There is no noticeable benefit of using a paper with a pore size below about 0.5 μm since the wicking of the plasma may be sufficiently retarded as to be undesirable. A substrate thickness of 50 to 200 μm is preferable with 130-160 μm being optimal. The thickness of the substrate is selected to have sufficient cross-sectional area to provide adequate sample for signal detection while utilizing as small a sample as possible. GE Filter Paper Grade 93 has proven to be a suitable substrate for demonstration of the invention.

The barrier functions to provide structural support for the substrate, inhibits oxidation of the coatings on the substrate, and isolates the location of sample introduction on the substrate to the vacancy described as the sample window elsewhere herein. A particularly preferred barrier comprises an adhesive on one side thereby allowing the test substrate to be formed by overlaying the barrier on the substrate with a vacancy remaining to function as the sample window. Transparent vinyl with a contact adhesive on one side, such as a contact paper, is particularly preferred for demonstration of the invention. Commercially available Duck Brand 1115496 Peel N′ Stick Laminate Adhesive Shelf Liner, 12-Inch×36-Feet, Clear is particularly suitable for demonstration of the invention.

The light emitter is not particularly limited herein with the understanding that the emitted light must be absorbed by the indicator and therefore a light which emits at a wavelength consistent with the peak absorption of the indicator is preferred. For ABTS a violet light is preferred and more preferably a wavelength of about 420 nm is preferred. Light emitting diodes (LED's) are preferred due to their low cost, low energy consumption, relatively monochromic light, ready availability and they are DC powered. A 9V battery is a suitable power source for demonstration of the invention. Furthermore, LED's are easily obtained with a wavelength of choice which aids in adaptation to the dye of choice. With ABTS a violet emitting LED is most preferred.

The sensor is not particularly limited herein with the understanding that the sensor is sensitive to the wavelength of light emitted by the LED and preferably the sensitivity at the wavelength of light emitted by the LED is sufficient for accurate determination of the transmitted light. Depending on the sensor employed signal amplification may be appropriate, especially, when a small umbra or a thin substrate is employed. Photodiodes are suitable for demonstration of the invention with external amplification being suitable for most instances. LOG amplifiers can be employed but are not preferred due to cost considerations. A particular advantage of the inventive system, particularly with two LEDS, is that the meter calibrates itself with every reading. Other meters have calibration solution and are recommended to be calibrated every 6 weeks or so, but the solution is often lost or forgotten. The inventive sensor preferably has built-in circuitry to help maintain accuracy even in changing environmental factors. The sensor is chosen to be able to detect a color change in the indicator consistent with a glucose level over a range of 0 to 350 mg/dL with an error of no more than 15% when above 75 mg/dL and within 10 mg/dL when below 75 mg/dL.

A particular advantage of the instant invention is the lack of preservatives. The system can be prepared and used within a short period of time thereby eliminating the need for preservatives. The active components, and more specifically the glucose oxidase, peroxidase and indicator, are preferably provided as a powder or lypholized powder. In the powder form these materials are stable for a very long time thereby eliminating the need for preservatives. Whereas preservatives are typically necessary the instant invention can be used without preservatives and preferably with no more than 2 wt % preservative. As a powder the glucose oxidase, peroxidase and indicator have a very long shelf life and can be stored for four years without detriment. Once formulated as a liquid, and placed into the cartridge, the solutions can be stored for four weeks without detriment. Once printed the test substrate can be stored for six weeks without detriment. It is preferable to use the test strip within four weeks and the liquids in the cartridge within three days.

The printer is not limited herein, however, it is preferable that the printer have a spongeless cartridge as cartridges containing sponges have been demonstrated to be more difficult to clean for subsequent use. It is also preferable to utilize a printer with separate cartridges, instead of a single cartridge with multiple chambers, for simplicity of operation. An Epson Workforce30 printer has proven to be suitable since this printer utilizes separate cartridges for each color, and the cartridges are spongeless. An additional benefit is that there are third party suppliers that provide these cartridges empty which eliminates the need to clean the cartridge of ink prior to loading it.

An exemplary embodiment illustrating the amounts of each component are reproduced in Table 1 as would be suitable for use with an Epson Workforce30 printer.

TABLE 1

| Powder | | | Amount per |
|---|---|---|---|
| Name | Active units/g | Amount/mL | cartridge |
| Glucose | 100,000-250,000 | 0.00064-0.0016 g | 0.064-0.016 g |
| Oxidase | | | |
| Horseradish | 250,000-330,000 | 0.0067-0.008 g | 0.067-0.08 g |
| Peroxidase | | | |
| ABTS | NA | 0.0002-0.002 g | 0.002-0.02 g |

The invention has been described with reference to the preferred embodiments without limit thereto. One of skill in the art would realize additional embodiments and improvements which are not specifically set forth herein but which are within the scope of the invention as more specifically set forth in the claims appended hereto.

Claimed is:

1. A system for measuring glucose in blood comprising:
a substrate comprising a sample umbra and a control umbra;
a printer capable of printing coatings on said substrate thereby forming a printed substrate wherein:
a first coating of said coatings comprises glucose oxidase and wherein said first coating is in said sample umbra but not in said control umbra;
a second coating of said coatings comprises peroxidase and wherein said second coating is in said sample umbra and said control umbra; and
a third coating of said coatings comprises indicator and wherein said third coating is in said sample umbra and said control umbra;
a barrier capable of encasing at least a portion of said coated substrate except for a sample window and wherein when said blood is applied to said sample window plasma of said blood wicks into said substrate exclusive of red blood cells of said blood such that said plasma enters said sample umbra and said control umbra; and
a detector capable of measuring a color change of said indicator in said sample umbra and said control umbra.

2. The system for measuring glucose in blood of claim 1 wherein said substrate comprises paper with a pore size of at least 0.5 μm to no more than 10 μm.

3. The system for measuring glucose in blood of claim 1 wherein said peroxidase is horseradish oxidase.

4. The system for measuring glucose in blood of claim 3 wherein said horseradish peroxidase is selected from the group consisting of horseradish peroxidase Type I, horseradish peroxidase Type II and horseradish peroxidase Type VI.

5. The system for measuring glucose in blood of claim 1 wherein said indicator is selected from the group consisting of O-dianisidine; 3,3′,5,5′-tetramethylbenzidine; o-phenylene-diamine; 5-aminosalicylic acid and 2,2′-azino-bis(3-ethylbenzothizoline-6-sulphonic acid).

6. The system for measuring glucose in blood of claim 5 wherein said indicator is 2,2′-azino-bis(3-ethylbenzothizoline-6-sulphonic acid).

7. The system for measuring glucose in blood of claim 1 wherein said detector comprises an LED.

8. The system for measuring glucose in blood of claim 1 wherein said printer comprises cartridges wherein a first cartridge of said cartridges comprises said glucose oxidase, a second cartridge of said cartridges comprises said peroxidase and a third cartridge of said cartridges comprises said indicator.

9. The system for measuring glucose in blood of claim 1 wherein at least one of said glucose oxidase, said peroxidase of said indicator is a solid.

10. The system for measuring glucose in blood of claim 9 further comprising a liquid container for mixing with at least one of: said glucose oxidase to form said first coating; said peroxidase to form said second coating or said indicator to form said third coating.

11. The system for measuring glucose in blood of claim 9 wherein said solid is in a cartridge for said printer.

12. The system for measuring glucose in blood of claim 1 wherein said barrier comprises vinyl.

13. A method for testing glucose in blood comprising:
providing a substrate comprising a sample umbra and a control umbra;
providing a printer capable of applying coatings on said substrate wherein said printer comprises cartridges wherein:
a first cartridge of said cartridges comprises a first liquid wherein said first liquid comprises glucose oxidase;
a second cartridge of said cartridges comprises a second liquid wherein said second liquid comprises peroxidase; and
a third cartridge of said cartridges comprises a third liquid wherein said third liquid comprises indicator;
forming a printed substrate by:
applying said first liquid to said substrate thereby forming a first coating wherein said first coating is in said sample umbra but not in said control umbra;
applying said second liquid to said substrate thereby forming a second coating wherein said second coating is in said sample umbra and in said control umbra;
applying said third liquid to said substrate thereby forming a third coating wherein said third coating is in said sample umbra and in said control umbra;
encasing said printed substrate with a barrier except for a sample window wherein said barrier covers said sample umbra and in said control umbra thereby forming an encased substrate;
applying said blood to said encased substrate at said sample window;
measuring a sample color change in said sample umbra and in a control color change in said control umbra; and
subtracting said control color change from said sample color change to provide a resulting color change wherein said resulting color change is proportional to said glucose in said blood.

14. The method for testing glucose in blood of claim 13 wherein at least one of said glucose oxidase, said peroxidase or said indicator is a solid and further comprising adding a liquid to said solid prior to said forming said coated substrate.

15. The method for testing glucose in blood of claim 14 wherein said solid is in a cartridge of said cartridges.

16. The method for testing glucose in blood of claim 13 wherein said substrate has a thickness of at least 50 μm to no more than 200 μm.

17. The method for testing glucose in blood of claim 13 wherein said peroxidase is horseradish oxidase.

18. The method for testing glucose in blood of claim 17 wherein said horseradish peroxidase is selected from the group consisting of horseradish peroxidase Type I, horseradish peroxidase Type II and horseradish peroxidase Type VI.

19. The method for testing glucose in blood of claim 13 wherein said indicator is selected from the group consisting of O-dianisidine; 3,3′,5,5′-tetramethylbenzidine; o-phenylene-diamine; 5-aminosalicylic acid and 2,2′-azino-bis(3-ethylbenzothizoline-6-sulphonic acid).

20. The method for testing glucose in blood of claim 19 wherein said indicator is 2,2′-azino-bis(3-ethylbenzothizoline-6-sulphonic acid).
